(19) United States
(12) Patent Application Publication
(10) Pub. No.: US 2024/0238336 A1
(43) Pub. Date: Jul. 18, 2024

(54) COPPER NANOCLUSTERS, METHODS FOR OBTAINING SAME AND THE USE THEREOF IN THE TREATMENT OF MENKES DISEASE

(71) Applicants: UNIVERSITÉ DE LORRAINE, NANCY (FR); CENTRE HOSPITALIER REGIONAL UNIVERSITAIRE NANCY (CHRU), NANCY (FR)

(72) Inventors: Ariane BOUDIE, SAINT-MAX (FR); Igor CLAROT, EINVAUX (FR); François FEILLET, DOMBASLE SUR MEURTHE (FR)

(73) Assignees: UNIVERSITÉ DE LORRAINE, NANCY (FR); CENTRE HOSPITALIER REGIONAL UNIVERSITAIRE NANCY (CHRU), NANCY (FR)

(21) Appl. No.: 18/558,581

(22) PCT Filed: May 10, 2022
(86) PCT No.: PCT/FR2022/050896

§ 371 Date: Nov. 2, 2023

(30) Foreign Application Priority Data
May 11, 2021 (FR) FR2105001

(51) Int. Cl. A61K 33/34; A61K 9/51; A61K 47/54; A61K 47/55; A61P 25/28

(52) U.S. Cl. CPC A61K 33/34; A61K 9/5123; A61K 9/5192; A61K 47/542; A61K 47/551; A61P 25/28

(57) ABSTRACT

Copper nanoclusters designated by the formula “CuNC@HisAc” or “CuNC@HisAcAsc”, wherein the copper nanoclusters: are covered on the surface thereof by a mixed layer including histidine (His), acetate ions (Ac) and possibly ascorbate ions (Asc); have a spherical shape; have a hydrodynamical diameter of 0.6-2.0 nm; have a metallic core with a diameter of 0.5-1.5 nm; have a temporal stability of 5-12 weeks in solution; have a temporal stability of at least 12 months in dried form; and have spectrophotometric properties, with a shoulder in the UV-Visible spectrum at 325±10 nm and a fluorescence spectrum with excitation wavelengths of 365-395 nm and emission wavelengths of 445-475 nm. Also, a method for preparation of the copper nanoclusters, and the use of the copper nanoclusters in a method treatment of pathologies related to a copper deficit, and in particular Menkes syndrome.

### FIELD

The present disclosure comes from the field of chemistry, and more specifically pharmaceutical chemistry. The invention relates to copper nanoclusters, processes for obtaining same and application thereof in the treatment of diseases involving a copper deficit, and more specifically in the treatment of Menkes syndrome.

### BACKGROUND

Menkes syndrome is a rare genetic disease whose transmission is related to the X chromosome (X 21.1), which primarily affects boys and which has an occurrence corresponding to one birth per 300,000 in Europe, or two births each year in France. Menkes syndrome follows a copper deficit in the patient. Copper is a key oligo-element entering into the composition of many key enzymes of metabolism and is therefore indispensable to the survival of the human body. In the case of Menkes syndrome, a copper transporter, the protein ATPase 7A is not functional. Consequently, copper supplied by food cannot be absorbed, which leads to a profound copper deficit in the body. Further, this transporter allows the passage of copper into the brain, which has the consequence that the administration of copper to the blood cannot cure these patients because that does not treat the neurological involvement of the syndrome. Beyond the profound psychomotor development delay, children with Menkes syndrome have a major hypotonia, epilepsy uncontrollable by anticonvulsant treatment, and episodes of hypothermia, hypotension, etc. There are also cutaneous-integumentary anomalies with a lack of pigmentation, pale and dry skin, and fragile, dry and abnormal hair (Kinky hair). There may also be vascular anomalies causing subdural hematomas and also osseous mineralization problems. Currently, Menkes syndrome remains incurable and death in general occurs early in childhood (lifespan less than five years).

The only available therapeutic option relates to the use of a copper histidine complex (copper histidinate) provided by the Paris Agence Générale des Equipements et Produits de Santé (AGEPS) part of Assistance Publique-Hêpitaux de Paris (AP-HP). This product is administered parenterally (subcutaneous). This treatment makes the copper available in the blood compartment, but as explained above, does not allow its passage to the brain, and therefore does not cure the neurological involvement of this syndrome. The document “Sr Sheela et al, Clinical Genetics, Wiley-Blackwell Munksgaard, Dk, vol. 68, No. 3, Aug. 11, 2005, pages 278-283” is a study from India done on a 15-month-old Indian child, who had Menkes syndrome, and who was treated by daily subcutaneous injections of copper histidine for two and a half years. With the treatment, the epilepsy crises were eliminated and an improved pigmentation of the skin and hair followed. However, the child continued to manifest severe developmental delays.

There is currently no satisfactory solution for improving the life expectancy of children with Menkes syndrome and/or for improving their quality of life. The present disclosure aims to improve the situation because it proposes a solution aiming in particular to remedy the aforementioned disadvantages.

The inventors had the original idea of proposing the use of copper nanoclusters in the treatment of Menkes syndrome. In fact, to the knowledge of the inventors, no one had yet thought of using copper nanoclusters in the treatment of this syndrome. In this respect, the inventors have developed an original method for synthesis of copper nanoclusters. Various methods of synthesis have been reported in the literature for getting nanoclusters, and in particular copper nanoclusters. Thus, in the document by Hu Yanling et al. (“Microchimica Acta, Springer Vienna, Vol. 186, no. 1, Dec. 7, 2018, p. 1-8”) copper nanoclusters were synthesized from histidine, copper chloride and ascorbic acid. These copper nanoclusters, described as having very good fluorescence properties, have a spherical shape and a diameter of about 3 to 4 nm, where said diameter is evaluated by transmission electronic microscope. In the document CN 105,965,028 platinum, silver, gold and copper are described for their use in the detection of iron ions by fluorescence/luminescence. Synthesis of copper clusters from histidine, copper acetate and ascorbic acid is in particular described in this document; the clusters have an average size of 1.9 nm where said size is measured by transmission electron microscopy. This document is silent about the stability of the resulting clusters.

### SUMMARY

The present disclosure proposes copper nanoclusters for use thereof in the treatment of Menkes syndrome. In fact, the copper nanoclusters that are the subject of the present invention can pass the blood-brain barrier, and therefore to provide copper to the brain of the patients. Further, at the doses currently used on mouse models, they do not accumulate in organs such as the liver, spleen, kidneys or lungs. The copper nanoclusters from the invention have several more advantageous properties which will be described in more detail below.

The nanoclusters from the invention, because of the advantageous properties thereof, are therefore promising candidates for improving the quality of life and the life expectancy of patients with Menkes syndrome.

Another goal of the invention is to propose a simple and quick method for synthesis of the nanoclusters from the invention.

The subject of the present invention is copper nanoclusters characterized in that they:
are covered on the surface thereof by a mixed layer comprising histidine (His), acetate ions (Ac) and possibly ascorbate ions (Asc);
have a spherical shape;
have a hydrodynamical diameter ranging from 0.6 to 2.0 nm, preferably 1.3 nm;
have a metallic core with a diameter ranging from 0.5 to 1.5 nm, preferably 1.0 nm;
have a temporal stability ranging from 5 to 12 weeks, when the nanoclusters are in liquid form;
have a temporal stability of at least 12 months, and preferably of 12 to 18 months, when the nanoclusters are in dried form;
have spectrophotometric properties, with a shoulder in the UV-Visible spectrum at 325±10 nm and a fluorescence spectrum with excitation wavelengths ranging from 365 to 395 nm and emission wavelengths ranging from 445 to 475 nm;
said copper nanoclusters are designated by the formula “CuNC@HisAc” or by the formula “CuNC@HisAcAsc.” The copper nanoclusters that are the subject of the invention are metal nanoclusters. A metal nanocluster consists of the association of dozens of metal element atoms (copper in the case of the invention) with a metal core diameter less than or equal to 2.0 nm (nanometer).

The CuNC@HisAc nanoclusters from the invention are made up of a copper core covered/covered up/surrounded by a mixed layer/crown of histidine, acetate ions and possibly ascorbate ions. In the application, the terms “crown” or “layer” comprising histidine, acetate ions and possibly ascorbate ions may be used without distinction. The term “mixed” is used to indicate that the crown or the layer which surrounds the copper core comprises together histidine, acetate ions and possibly ascorbate ions. Similarly, the verbs “cover/cover-up/surround” may be used without distinction for indicating that the copper core comprises over the entire surface thereof a layer/crown of histidine, acetate ions and possibly ascorbate ions. As a whole, the CuNC@HisAc copper nanocluster has a spherical shape.

In the meaning of the invention, the formula “CuNC@HisAc” designates a nanocluster made up of a copper metal core covered by a mixed layer comprising histidine, acetate ions and possibly ascorbate ions. Also, the formula “CuNC@HisAc” without other indications in the present application also encompasses the formula “CuNC@HisAcAsc,” which is a specific embodiment of a copper nanocluster from the invention, specifically a nanocluster constituted of a copper metal core covered with a mixed layer comprising histidine, acetate ions and ascorbate ions.

The mixed crown comprising histidine, acetate ions and possibly ascorbate ions confers in particular to the nanoclusters from the invention a great stability and a low reactivity.

A “low reactivity” means a low breakdown in particular related to oxygen (for example molecular oxygen in the air).

According to a particularly advantageous embodiment of the invention, the mixed layer/crown surrounding the copper metal core comprises histidine, acetate ions and ascorbate ions. The copper nanoclusters from the invention thus advantageously comprise three ligands on the surface of the copper core, specifically histidine, acetate and ascorbate. These histidine ligands, acetate ions and ascorbate ion are bound to the copper metal core by coordinate bonds. The stability of the nanoclusters from the invention indicates a maintenance of the structure and properties of the nanoclusters over time at a storage temperature of 4° C.

Maintenance of the structure means in particular that the composition of the nanocluster (metal core surrounded by the mixed layer/crown as defined above), the shape thereof and diameter thereof (both metal core and hydrodynamic) are retained over time.

“Liquid form” of the copper nanoclusters designates a solution or a liquid mixture of copper nanoclusters. The 5 to 12-week stability mentioned above relates to copper nanoclusters in liquid form.

“Dry form” is understood to mean a solid form which may be reduced to powder if necessary.

The 12 to 18-month stability mentioned above relates to copper nanoclusters in dry form.

Depending on their form (liquid or solid), their stability over time will therefore be different.

The nanoclusters from the invention have spectrophotometric properties, in particular fluorescence, which are characteristic of this scale, specifically a metal core diameter less than or equal to 2 nm, which is intermediate between the molecule and the nanoparticle.

The metal core diameter or metal diameter designates, as its name indicates, the diameter formed by the copper metal.

The hydrodynamic diameter considers the diameter of the copper core and the layers/crown thereof comprising histidine, acetate ions and possibly ascorbate ions. The hydrodynamic diameter therefore designates the diameter of the CuNC@HisAc copper nanocluster from the invention.

It can be observed that the nanoclusters from the invention have nearly equivalent metal core and hydrodynamic diameters (respectively 1.0 and 1.3 nm).

In the present application, the nanoclusters of the invention are designated without distinction by “nanoclusters,” “copper nanoclusters” and “CuNC@HisAc nanoclusters,” where it is understood that “CuNC@HisAc nanoclusters” encompasses the specific embodiment “CuNC@HisAcAsc nanoclusters.”

According to an embodiment of the invention, the copper nanoclusters come in liquid form or dry form.

The dry form of the nanoclusters is advantageous in particular in that it allows easy storage, preservation and transport of the nanoclusters from the invention.

According to an advantageous embodiment, the copper nanoclusters from the invention also characterized in that they have at least one of the following characteristics:
they are able to pass through the blood-brain barrier;
they have a good bioavailability;
they are biocompatible;
they are biodegradable;
they can be freeze-dried;
they are not toxic for the human body;
they do not accumulate in organs such as the liver, spleen, kidneys or lungs.

According to an advantageous embodiment, the copper nanoclusters from the present invention have all of the characteristics described above.

The fact that the nanoclusters from the invention are not sequestered in said organs is in particular due to their small size (hydrodynamic diameter less than or equal to 2.0 nm). The size of the nanocluster from the invention allows a longer circulation in the blood, compared to larger size compounds.

The nanoclusters from the invention have surface properties that make them capable of passing through the blood-brain barrier by using the cerebral-blood capillary-transport systems. Further, histidine serves in particular to help address the nanocluster to the brain because histidine is transformed into histamine there.

“Good bioavailability” means that when the copper nanoclusters are administered subcutaneously, they do reach the general circulation and are well distributed to the target organs.

“Biocompatibility is understood to mean the fact that the copper nanoclusters are accepted by the various organs of the body and allow a restoration of the biological functions related to cuproproteins without being toxic for these organs.

The fact that the nanoclusters are biodegradable means that their breakdown releases substances which are metabolized or eliminated from the body without difficulty (copper, histidine, acetate and possibly ascorbate).

The fact that the nanoclusters can be freeze-dried serves in particular to store, preserve and transport them easily. The fact that the nanoclusters can be freeze-dried is attributable to the fact that the nanoclusters are perfectly stable. The stability of the nanoclusters is such as defined above.

The advantageous properties of the nanoclusters from the invention are in particular due to the original combination of the constituents thereof, specifically copper, histidine, acetate ions and possibly ascorbate ions. To the knowledge of the inventors, copper nanoclusters have never been described to date comprising a mixed crown/layer of histidine, acetate ions and possibly ascorbate ions surrounding a copper metal core, and which have the advantageous properties described above.

The object of the present invention is also a method for preparation of copper nanoclusters such as described above, characterized in that it comprises the following steps:
reaction between copper (II) acetate and histidine in order to get a mixture of copper acetate and histidine, where the molar ratio of histidine to copper (II) acetate is greater than or equal to 50, and preferably goes from 50 to 200;
reaction between the mixture of copper acetate and histidine with ascorbic acid in order to get a mixture of copper acetate, histidine and ascorbic acid, where the molar ratio of ascorbic acid/copper (II) acetate is greater than or equal to 130, and preferably goes from 130 to 700;
recovery of copper nanoclusters covered on the surface thereof by a mixed layer comprising histidine, acetate ions and possibly ascorbate ions.

According to an advantageous embodiment of the invention, the copper nanoclusters obtained at the outcome of the method are covered on the surface thereof with a mixed layer comprising histidine, acetate ions and ascorbate ions.

The molar ratio such as defined above, respectively between histidine and copper acetate, and between ascorbic acid and copper acetate, are important in this sense that they allow the histidine, acetate and ascorbate ligands to bind to the copper metal core. This way nanoclusters are obtained comprising three ligands on the surface of the copper core, these ligands being bound to the surface of the copper core by coordination bonds.

Ascorbic acid is a reducer. The reaction between the copper acetate and histidine mixture with ascorbic acid is more specifically a reduction reaction of the copper acetate and histidine mixture with ascorbic acid. The ascorbic acid serves in particular to get copper nanoclusters without any toxicity.

The present invention results in particular from the unexpected discovery by the inventors that the original combination of reagents used, specifically copper acetate, histidine and ascorbic acid, and in the proportion such as defined above, serves to get copper nanoclusters with particularly advantageous properties.

The excellent stability of the nanoclusters from the invention is an example.

According to an embodiment of the invention, the copper nanoclusters may more specifically be prepared according to the “solution” protocol or according to the “solid-phase” protocol. Each of these two synthetic pathways conforms to the methods described above.

### Solution Protocol

According to an advantageous embodiment of the invention, the method for preparation as defined above is more specifically characterized in that it is done under inert gas and in that:
the copper acetate is in solution form and the histidine is in powder form;
a solution of copper acetate and histidine is prepared by adding histidine to the copper acetate solution;
the copper acetate and histidine solution is adjusted to a pH value ranging from 11 to 13, and is preferably 12;
the ascorbic acid is in powder form;
a solution of copper acetate, histidine and ascorbic acid is prepared by addition of ascorbic acid to the copper acetate and histidine solution whose pH was adjusted to the aforementioned values;
the copper acetate, histidine and ascorbic acid solution is stirred for 2 hours to 6 hours, and preferably 4 hours, at a temperature ranging from 35° C. to 45° C., and preferably 40° C.;
a solution comprising CuNC@HisAc copper nanoclusters is obtained at the outcome of the preceding stirring step;
the solution comprising the copper nanoclusters could be dialyzed in order to get a purified copper nanocluster solution;
the solution comprising the copper nanoclusters, possibly dialyzed, could be freeze-dried in order to get dry-form copper nanoclusters.

The solution comprising the copper nanoclusters, possibly dialyzed, has a stability over time ranging from 5 to 12 weeks at a storage temperature of 4° C.

Dialysis serves in particular to eliminate anything which is not bound to the copper metal core, like, for example, a possible excess of histidine or ascorbic acid, or even residual copper which could be present in the nanocluster solution. The layer comprising histidine, acetate ions and possibly ascorbate ions is bound to the copper metal core by coordinate bonds.

The dry-form of the copper nanoclusters, obtained at the outcome of freeze-drying, has a stability over time of at least 12 months, preferably 12 to 18 months, at a storage temperature of 4° C. under nitrogen.

The dry form of copper nanoclusters may be reconstituted at any time, by mixture in a reconstitution solvent, for example purified water. “To reconstitute/reconstitution” is understood to mean the simple mixing operation of the dry or freeze-dried form with a solvent.

The analysis of the copper nanocluster solution, obtained at the outcome of reconstitution of the dry form, shows that the copper nanoclusters have all the properties defined above and are therefore exactly the same as those obtained directly at the outcome of the method for their preparation.

The copper nanocluster solution, obtained at the outcome of the reconstitution the dry form, has a stability over time ranging from 5 to 12 weeks at a storage temperature of 4° C.

The method for preparation as defined above is also characterized in that it further comprises at least one characteristic selected from:
the inert gas is nitrogen;
the copper acetate solution is prepared by addition of copper acetate to ultrapure filtered water;
the concentration of the copper acetate solution ranges from 0.5 to 5.0 mM;
the concentration of histidine is greater than the concentration of the copper acetate solution;
the pH of the copper acetate and histidine solution is adjusted using sodium hydroxide;
the solution comprising the copper nanoclusters, possibly dialyzed, has a copper concentration ranging from 16 to 160 μg/mL;
the solution comprising the copper nanoclusters, possibly dialyzed, is freeze-dried in order to get dry-form copper nanoclusters.

According to an advantageous embodiment, the method from the present invention has all of the characteristics described above.

### Solid-Phase Protocol

According to another advantageous embodiment of the invention, the method for preparation as defined above is more specifically characterized in that:
the copper acetate is in powder form and the histidine is in powder form;
a powdered mixture of copper acetate and histidine is obtained by mixing each of the powders of copper acetate and histidine;
the powdered copper acetate and histidine mixture is milled until obtaining a homogeneous color powdered mixture;
the homogeneous powdered copper acetate and histidine mixture is placed in a reaction vessel;
the ascorbic acid is in powder form;
ascorbic acid is added to the reaction vessel comprising the homogeneous powdered copper acetate and histidine mixture;
the resulting powdered copper acetate, histidine and ascorbic acid mixture is stirred, then water is added drop by drop to the reaction vessel, where said water is filtered ultrapure water;
the reaction vessel is placed under an inert gas and protected from light;
the copper acetate, histidine, ascorbic acid and water mixture is kept under stirring in the reaction vessel for 16 to 36 hours, preferably for 24 hours;
a liquid mixture comprising CuNC@HisAc copper nanoclusters is obtained at the outcome of the preceding stirring step;
the liquid mixture comprising the copper nanoclusters could be dialyzed in order to get a purified liquid copper nanocluster mixture;
the liquid mixture comprising the copper nanoclusters, possibly dialyzed, could be freeze-dried in order to get dry-form copper nanoclusters.

The stability data described above for the copper nanoclusters, in liquid form or in dry form, obtained at the outcome of the solution protocol are valid for the copper nanoclusters in liquid or dry form obtained of the outcome of the solid-phase protocol.

The copper acetate powder used above is blue and the histidine powder is white. When these two powders are mixed, the desired result is the most homogeneous possible light-blue color.

The method for preparation as defined above is also characterized in that it further comprises at least one characteristic selected from:
the concentration of histidine is greater than the concentration of copper acetate;
the water added to the reaction vessel is filtered ultrapure water;
the inert gas is nitrogen;
the liquid mixture comprising the copper nanoclusters, possibly dialyzed, has a copper concentration ranging from 1600 to 4000 μg/mL;
the liquid mixture comprising the copper nanoclusters, possibly dialyzed, is freeze-dried in order to get dry-form copper nanoclusters.

The freeze-dried form is stored under nitrogen.

According to an advantageous embodiment, the method from the present invention has all of the characteristics described above.

According to another embodiment of the invention, the dry-form copper nanoclusters obtained at the outcome of the freeze-drying (according to the “solution” or “solid-phase” protocol) is stored under nitrogen, preferably in vials, and preferably at 4° C. Under such conditions, the copper nanocluster powder could be stored for at least 12 months, and preferably for 12 to 18 months, without alteration of the stability of the copper nanoclusters.

The reconstitution of the nanocluster powder at the end of this time shows that the nanoclusters are exactly the same as those obtained directly at the outcome of the preparation thereof (according to the “solution” or the “in-solid phase” protocol). In fact, the copper nanoclusters have all the properties defined above.

According to another aspect, the object of the present invention is also copper nanoclusters such as defined above or obtained according to the methods as defined above for use as a medication.

The object of the present invention is more specifically copper nanoclusters as defined above or obtained according to the methods as defined above for use in treatment of pathologies related to a copper deficit.

As example(s) of pathologies related to a copper deficit, Menkes syndrome can be more specifically cited.

The copper nanoclusters from the invention are more specifically intended for patients with an ATPase7A deficit.

Among the patients with an ATPase7A deficiency, patients with Menkes syndrome, specifically children, can be cited. According to an embodiment of the invention, the copper nanoclusters are therefore more specifically intended for pediatric use.

However, among the patients with an ATPase7A deficit, adult patients with the attenuated form of Menkes syndrome, specifically adult patients with an occipital horn syndrome (OHS) can also be mentioned. According to another embodiment of the invention, the copper nanoclusters are also suited for use in the treatment of occipital horn syndrome.

Again, an object of the invention is a pharmaceutical composition containing a therapeutically effective quantity of copper nanoclusters such as defined above or obtained according to the methods as defined above.

The quantity of copper nanoclusters could vary according to the mode of administration considered and the age and weight of the patient.

The copper nanoclusters or the pharmaceutical composition from the invention could have a form appropriate for being administered subcutaneously or intravenously. According to a preferred embodiment of the invention, the copper nanoclusters have a form suited for subcutaneous administration, preferably once per day.

According to an advantageous embodiment, the copper nanoclusters or the pharmaceutical composition from the invention have a form suited for administration via a subcutaneous pump.

As an example, the subcutaneous pump has been used for many years in the treatment of childhood diabetes. The medication is thus administered continuously, via a pump, that the patient wears on their body, and which administers the medication continuously through a subcutaneous catheter.

The invention also relates to a therapeutic treatment method for Menkes syndrome comprising the administration to a patient of a therapeutically effective quantity of copper nanoclusters or of a pharmaceutical composition such as defined above.

### BRIEF DESCRIPTION OF THE DRAWINGS

Other characteristics, details and advantages will appear upon reading the detailed description below, and analyzing the attached drawings, on which:

FIG. 1 is a schematic representation of a CuNC@HisAc copper nanocluster from the invention, made up of a copper metal core surrounded by a mixed crown comprising histidine and acetate.

FIG. 2 is a schematic representation of a CuNC@HisAcAsc copper nanocluster from the invention, made up of a copper metal core surrounded by a mixed crown comprising histidine, acetate and ascorbate.

FIG. 3 is an ion chromatography analysis of CuNC@HisAc copper nanoclusters, showing the presence of acetate ions on the surface of the copper metal core.

FIG. 4 is an infrared spectrophotometry analysis, showing the presence of ascorbate ions on the surface of the copper metal core.

FIG. 5 shows the hydrodynamic diameter (in nanometers) of CuNC@HisAc copper nanoclusters evaluated by dynamic light diffusion.

FIG. 6 shows the metal diameter (in nanometers) of CuNC@HisAc copper nanoclusters evaluated by transmission electron microscope.

FIG. 7 shows the hydrodynamic diameter (in nanometers) of CuNC@HisAc copper nanoclusters evaluated by Taylor dispersion.

FIG. 8 is a UV-Physical spectrum of CuNC@HisAc copper nanoclusters.

FIG. 9 is a fluorescence spectrum of CuNC@HisAc copper nanoclusters.

FIG. 10 shows the cerebral copper concentration (in μg/g) in mice as a function of the treatment (treated or untreated) and of the dose of copper nanoclusters received: WT (“Wild-type”), NT (untreated hemizygous mice), 4× 3×/week (hemizygous mice treated with the 4× copper nanocluster dose, injected three days per week) and 4× 5×/week (hemizygous mice treated with the 4× copper nanocluster dose, injected five days per week). For each group of mice, the letter n indicates the number of mice treated.

FIG. 11 shows the survival of the animals on a Kaplan-Maier curve as a function of the treatment (treated or untreated) and of the dose of copper nanoclusters received: WT (“Wild-type”), NT (untreated hemizygous mice), 1× (hemizygous mice treated with the 1× copper nanocluster dose, injected three days per week), 2× (hemizygous mice treated with the 2× copper nanocluster dose, injected three days per week), 4× 3×/week (hemizygous mice treated with the 4× copper nanocluster dose, injected three days per week) and 4× 5×/week (hemizygous mice treated with the 4× copper nanocluster dose, injected five days per week). For each group of mice, the letter n indicates the number of mice treated.

FIG. 12 shows a histological marker of copper by rhodanine staining of a section of kidney tissue coming from untreated hemizygous mice and hemizygous mice treated with a solution of copper and nanoclusters at a 2× dose (HE ×2).

FIG. 13 shows a histological marker of copper by 12 hodamine staining of a section of liver tissue coming from untreated hemizygous mice (HE NT) and hemizygous mice treated with a solution of copper and nanoclusters at a 2× dose (HE ×2).

FIG. 14 shows of the respiratory chain in the brain from untreated hemizygous mice (HE NT) and hemizygous mice treated with a solution of copper and nanoclusters at a 2× dose (HE 2×).

FIG. 15 shows the hydrodynamic diameter (in nanometers), evaluated by dynamic light diffusion, of copper clusters obtained according to the synthetic protocol described in the document CN 105,965,028. The hydrodynamic diameter was evaluated immediately after the synthesis of the copper clusters.

FIG. 16 shows the hydrodynamic diameter (in nanometers), evaluated by dynamic light diffusion, of copper clusters obtained according to the synthetic protocol described in the document CN 105,965,028. The hydrodynamic diameter was evaluated 24 hours after the synthesis of the copper clusters (storage at 4° C. in the dark).

FIG. 17 is a photo of three lots of copper clusters obtained according to the synthetic protocol described in the document CN 105,965,028 after 24 hours of storage at 4° C. in the dark.

### DETAILED DESCRIPTION

The drawings in the following description contain, for the most part, elements of a definite nature. They will therefore not only serve to make the present disclosure better understood, but also contribute to the definition thereof, as applicable.

### EXAMPLES

### Example 1

### Preparation of CuNC@HisAc Copper Nanoclusters

This example describes respectively the two possible synthetic pathways for preparing copper nanoclusters from the invention, specifically the “solution protocol” and the “solid-phase protocol.”

### 1) Solution Protocol

### Reagents Used

Copper (II) acetate [Cu(CH₃COO)₂], M=181.63 g/mol (CAS 142-71-2);
L(−)-Histidine, M=155.15 g/mol (CAS 71-00-1);
Ascorbic acid, M=176.12 g/mol (CAS 50-81-7);
1M solution of sodium hydroxide, M=40.00 g/mol (CAS 1310-73-2).

### Precautions to Take

The synthesis is done under an inert gas (nitrogen). The glassware is washed with aqua regia (one volume of 65% nitric acid, for two volumes of 37% hydrochloric acid).

Since the copper nanoclusters are intended to be studied in vivo, it is necessary to work under a clean hood and clean all the equipment used with 70% ethanol by volume. The ultrapure water used is filtered with a filter with 0.2 μm diameter pores.

### Preparation of a Copper Acetate Mother Solution

Quantity of 45 mg of copper acetate is placed in a 100 mL volumetric flask. Filtered ultrapure water is added up to the line on the volumetric flask. The result is a 2.5 mM concentration solution of copper acetate.

After completely dissolving, the copper acetate solution is transferred to a suitable container.

This solution may be stored for one month at 4° C.

Synthesis of Copper Nanoclusters Stabilized with Histidine

Quantity of 500 μL of copper acetate mother solution such as prepared in the previous step is added to a round-neck round-bottom flask which can contain up to 50 mL of solution. Then a quantity of 4500 μL of ultrapure filtered water is added to the round-bottom flask. The resulting copper acetate solution is called 1×.

2× and 4× copper acetate solutions are also prepared by placing a respective quantity of 1000 μL and 2000 μL of copper acetate mother solution in a round bottom flask and bringing up to 5000 μL with ultrapure filtered water.

The 1× copper acetate solution is stirred at 130 RPM with the multi-plate stirrer. A quantity of 39 mg of histidine is added to the copper acetate solution. The solution of copper acetate and histidine is stirred for 15 minutes. The solution takes on a bluish tint. After 15 minutes of stirring, the pH of the copper acetate and histidine solution is adjusted to 12 with 10 drops of 1M NaOH.

A quantity of 139 mg of ascorbic acid is added to the reaction mixture.

The round bottom flask (reaction vessel) is placed in a steam bath of 40° C. under stirring (speed set to 6) for four hours. The solution takes an orange tint.

The resulting copper nanocluster solution is kept cool, at 4° C.

The operations described above are repeated respectively with the 2× and 4× copper acetate solutions.

The copper concentrations obtained with copper acetate solutions from 1× to 10× for the copper nanocluster solutions go from 16 to 160 μg/mL.

The synthetic yield is 100%: there is no residual copper Cu²⁺ in the nanocluster solution.

The three copper nanocluster solutions, obtained respectively from a 1×, 2× and 4× copper acetate solution, are called in the following: 1×, 2× and 4× copper nanocluster solutions. The solutions may be freeze-dried.

### Dialysis of the Copper Nanoclusters

The copper nanoclusters resulting from the preceding step are purified by dialysis. A dialysis cell is prepared (X12 Float A-Lyzer G2 CE MWCO 100-500 D, Catalog number 1511160), and a 150 mL beaker is filled with 100 mL of filtered ultrapure water. The dialysis cell is filled with filtered ultrapure water using a Pasteur pipette. The dialysis cell is placed in the beaker with stirring (130 RPM). The cell is left to hydrate and wash for 1 hour.

The water is then replaced by a new volume of 100 mL filtered ultrapure water. The dialysis cell is emptied using a Pasteur pipette and then is filled with the 1× copper nanocluster solution, which is left under stirring all night (about 12 hours) at a temperature between 2 and 6° C.

The dialyzed 1× copper nanocluster solution is transferred to a suitable vial.

The same operations are repeated respectively with the 2× and 4× copper nanocluster solutions.

The copper concentrations of the dialyzed copper nanocluster solutions are identical to those of the undialyzed solutions, and range from 16 to 160 μg/mL (with copper acetate solutions from 1× to 10×).

The solutions may be freeze-dried.

### 2) Solid-Phase Protocol

### Reagents Used

The copper (II) acetate, L(−)-histidine and ascorbic acid are the same as used in the solution protocol. In the solid phase protocol, sodium hydroxide is not needed.

### Precautions to Take

The synthesis is done under inert gas (nitrogen) and protected from light. The glassware is washed with aqua regia (one volume of 65% nitric acid, for two volumes of 37% hydrochloric acid). The water used is ultrapure water filtered with a filter with 0.2 μm diameter pores.

Synthesis of Copper Nanoclusters Stabilized with Histidine

A quantity of 23 mg of copper acetate is weighed and then placed inside an agate mortar. A quantity of 1.7 g of histidine is then weighed. One volume of histidine powder for one volume of copper acetate powder is added taking care to fully mill the powders using the pestle until obtaining a mixture of homogeneous color and appearance. This operation is repeated so long as there is histidine. The final mixture of the two powders must have a light-blue color and the powder must be homogeneous. The mixture of the two powders is then transferred into a 50 mL single-neck round bottom flask (ground-joint neck NS 19/26) using a spatula. A quantity of 3 g of ascorbic acid is weighed and transferred to the round-bottom flask. An olive shape magnetic stir is placed in the bottom of the flask. A quantity of 5 mL of ultrapure water is filtered using a 5 mL plastic syringe and is added drop by drop into the reaction vessel. A liquid mixture results.

The reaction vessel is closed using a folding-skirt stopper (19.4 mm diameter) and is placed under nitrogen without creating excess pressure by means of a balloon. The reaction vessel is surrounded with aluminum foil and is then placed under stirring (200 RPM) for the length of the reaction. Before the end of the reaction, 24 hours is needed. At the end of the reaction, the resulting product, which is found in the form of a liquid, has a yellow color. The resulting liquid, comprising copper nanoclusters, has a copper concentration of 1600 μg/mL.

This liquid is then transferred to a suitable plastic vial (the final volume is a little more than 5 mL). The liquid comprising the copper nanoclusters is then stored cool, or transferred to the freeze dryer. The freeze-drying is done by 1 mL fractions. After freeze-drying, the content of the vial (which comprises the copper nanoclusters in dry form) is placed under nitrogen and is stored at 4° C. The dry form of copper nanoclusters may be reconstituted at any time in 1 mL purified water. The resulting copper nanocluster solution is stored under nitrogen.

### Example 2

### Characterization of Copper Nanoclusters (in Solution or in Dry Form and Returned to Solution

The copper nanoclusters as obtained an example 1 are characterized for their structure, size, spectrophotometric properties and stability.

### Structure of the Copper Nanoclusters

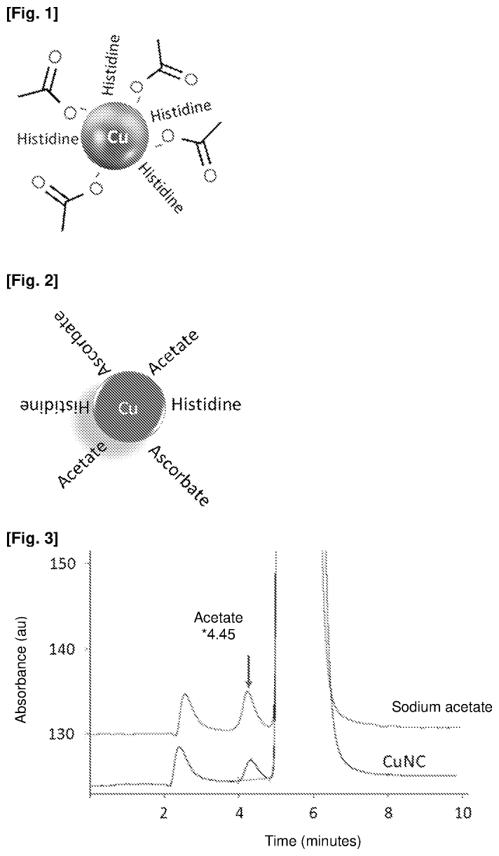

The copper nanoclusters from the invention have a spherical shape. They are more specifically made up of a copper metal core covered with a mixed crown comprising histidine, acetate ions and possibly ascorbate ions. FIG. 1 is a schematic representation of a CuNC@HisAc copper nanocluster from the invention, specifically a nanocluster which comprises on the surface of the copper core a mixed crown comprising histidine and acetate ions. FIG. 2 is a schematic representation of a specific embodiment, in particular a CuNC@HisAcAsc copper nanocluster comprising on the surface of the copper core a mixed crown comprising histidine, acetate ions and possibly ascorbate ions.

The presence of acetate ions on the surface of the copper nanoclusters was demonstrated by purification by steric exclusion chromatography and then ionic chromatographic quantification.

The copper nanoclusters from the 1× dialyzed solution as obtained from example 1 section 1 (“Solution Protocol”) are destroyed (total dissolution and returned to the various elements making up the structure of the nanoclusters) by a chemical method (dissolving in a concentrated acid (HCl) and then a concentrated base (NaOH)) and then are analyzed by ionic chromatography with comparison to a sodium acetate control (retention of ions composing the nanocluster on a cationic exchange resin, separation, identification and quantification (external calibration) of the acetate ions by conductivity measurement with use of a suppressor)).

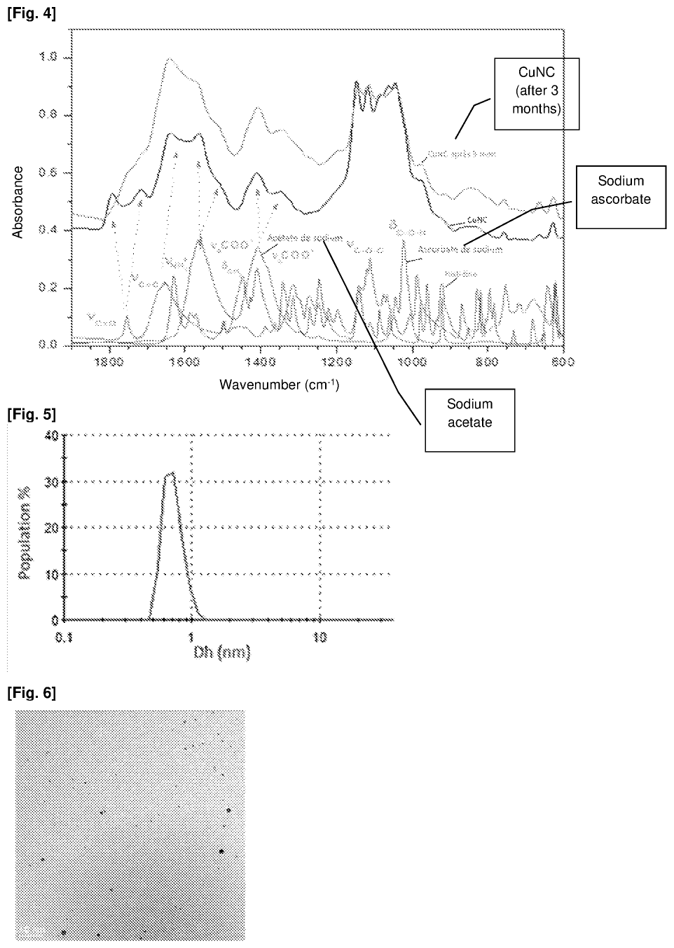

The results obtained with the ionic chromatographic analysis are shown in FIG. 3. The peak at 4.45 minutes associated with the acetate ions (control, see top trace) is found in the copper nanoclusters (see bottom trace).

The presence of ascorbate ions on the surface of the nanoclusters was demonstrated by infrared spectrophotometric analysis after purification of the nanoclusters in solution by stearic exclusion chromatography.

The results obtained with the infrared spectrophotometric analyzer are shown in FIG. 4. The characteristic wavelengths of the acetate ions from 1400 to 1500 cm⁻¹ are found during the analysis of the nanoclusters.

### Size of the Copper Nanoclusters

The hydrodynamic diameter (Dh) and/or metallic diameter of the copper nanoclusters was evaluated by dynamic light diffusion (FIG. 5) (173° angle, 530 nm laser, 25° C. temperature on Malvern Nanosizer), by transmission electronic microscopy (FIG. 6) (deposit on nickel grates, observation under beams operating at 200 kV (LaB6 cathode) Philips CM 200) and analyzed by Taylor dispersion (FIG. 7). The analysis method by dispersion consists of injecting a band of solute in an open capillary tube (50 μm) and mobilizing it under the influence of the hydrodynamic flow (1 psi positive pressure, parabolic velocity profile). The principal for the determination of the hydrodynamic radius rests upon the Taylor-Aris relation which establishes the link between the spread of the solute peak (modeled by a Gaussian) and the molecular diffusion coefficient.

The results obtained for the evaluation of the hydrodynamic diameter (Dh) of the copper nanoclusters by dynamic light diffusion are illustrated in FIG. 5.

The results obtained for the evaluation of the metal diameter of the copper nanoclusters by transmission electron microscopy are illustrated in FIG. 6. A majority population with an average diameter of less than 1.0 nm is observed.

The results obtained for the evaluation of the hydrodynamic diameter (Dh) of the copper nanoclusters by Taylor dispersion are illustrated in FIG. 7.

It follows from these pictures that the average hydrodynamic diameter, just like the metal diameter, of the CuNC@HisAc copper nanoclusters is less than 1.0 nm. The characteristics listed above relating to the copper nanoclusters concern more specifically copper nanoclusters covered on their surface with histidine, acetate ions and ascorbate ions.

### Spectrophotometric Properties

The spectrophotometric properties of copper nanoclusters were evaluated by UV-Visible spectroscopy and by fluorescence spectroscopy.

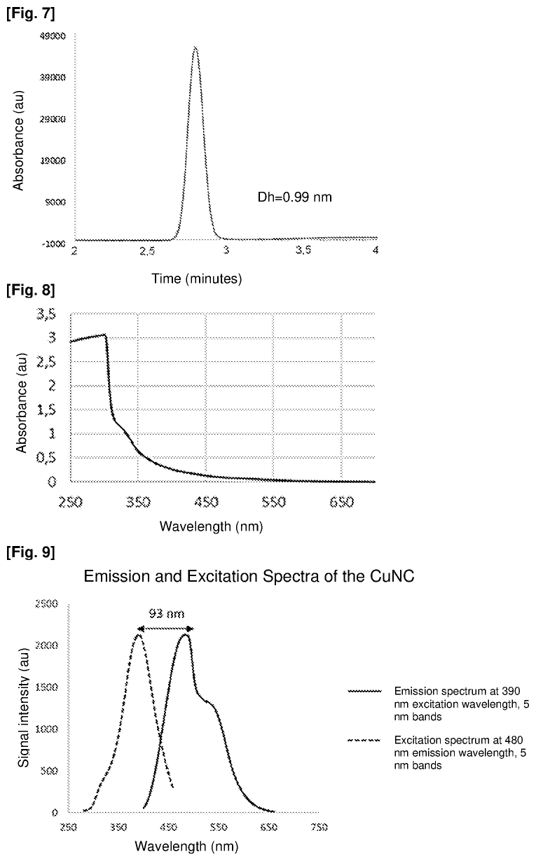

The results obtained with UV-Visible spectroscopy are shown in FIG. 8. It is observed that there is a shoulder on the of UV-visible spectrum at 325±10 nm which confirms the existence of the nanoclusters.

The results obtained with fluorescence spectroscopy are shown in FIG. 9. Fluorescence of copper nanoclusters exists with an excitation and emission wavelength of respectively 380±15 nm and 460±15 nm which confirms the existence of the nanoclusters.

The copper nanoclusters from the invention have optical properties, in particular fluorescence, characteristic of this intermediate scale between molecule and nanoparticle.

### Stability of the Copper Nanoclusters

The stability of copper nanoclusters was evaluated by dynamic light diffusion (angle 173°, laser 530 nm, temperature 25° C. on Malvern Nanosizer).

The analyses were done on the samples obtained at the outcome of the solution protocol and at the outcome of the solid-phase protocol.

More specifically, relating to the solution protocol, the analyses were done on copper nanocluster solutions 1×, 2× and 4×.

Relating to the solid-phase protocol, the analyses were done on the freeze-dried samples and on the solutions reconstituted after freeze-drying, stored or not under nitrogen. The stability of the nanoclusters is preserved in an environment free of molecular oxygen.

The results are not the same depending on whether the copper nanoclusters are stored in liquid form or solid form.

For the synthetic protocol in solution, the stability of the copper nanoclusters in solution is observed ranging up to:
140 days for copper nanoclusters prepared from a copper acetate solution 1× and 2×;
40 days for copper nanoclusters prepared from a copper acetate solution 4×.

For the solid-phase synthetic protocol, the stability of the freeze-dried copper nanoclusters is observed for at least 12 months for storage under nitrogen at 4° C. The measurement to the hydrodynamic diameter of the copper nanoclusters at 12 months of storage is 1.6 nm.

### Example 3

### In Vivo Tests in Mice

### Overview

Tests were done in vivo on the ATP7A-Mo^{blo} mouse model, which has a spontaneous mutation of the gene for Menkes syndrome, the ATP7A gene coding for ATPase 7A. The gene for Menkes syndrome is carried on the X chromosome. Heterozygous females are viable and fertile. As for the males, they are either healthy (normal X wild allele) or hemizygous (carrier of the mutation on the X chromosome) and therefore have Menkes syndrome. The majority of hemizygous animals have a defective elastin crosslinking level and also a collagen deficit, which explains early ruptures of aortic aneurysms and pulmonary emphysema and which means a mortality around five weeks of life. Copper absorption in the intestine is reduced 64% and copper concentration in the liver is reduced 56%. Males also have a cerebral noradrenaline deficit, which is caused by the cerebral dopamine-beta hydroxylase deficit, which is a copper-dependent enzyme.

Heterozygous females have a moderate phenotype with irregular hypopigmented spots on a coat with alternating dark zones and light zones. They have a normal metabolism and behavior, and are therefore capable of reproducing.

Healthy males have a black coat whereas hemizygous males have a light coat, without marks.

Further, the hemizygous males have a low weight, are generally small and sometimes have deformed posterior limbs.

### Tests Done

The copper nanocluster solutions 1×, 2× and 4× such as resulting from Example 1 (solution protocol) were respectively injected into male mice by subcutaneous injection in the back of the neck. Five groups of animals aged from five weeks (defined below) were studied.
1) WT Group: wild mice (“Wild Type”) (healthy males), who do not have a mutation on the ATP7A gene in the X chromosome, were treated at the beginning of the protocol to evaluate a possible toxicity of the excipient used to administer the copper nanocluster.
2) HE NT Group: the hemizygous mice (HE), which have an ATP7A gene mutation present on the X chromosome, are untreated, and serve as a control.
3) HE 1× Group: hemizygous mice are treated with a copper nanocluster solution (initial dose: 1×), which was administered three days per week.
4) HE 2× Group: hemizygous mice are treated with a 2× copper nanocluster solution (1× dose doubled), which was administered three days per week.
5) HE 4× Group: hemizygous mice are treated with a 4× copper nanocluster solution (1× dose quadrupled), which was administered either three days or five days per week.

### Cerebral Copper Assay and Hemizyqous Mice

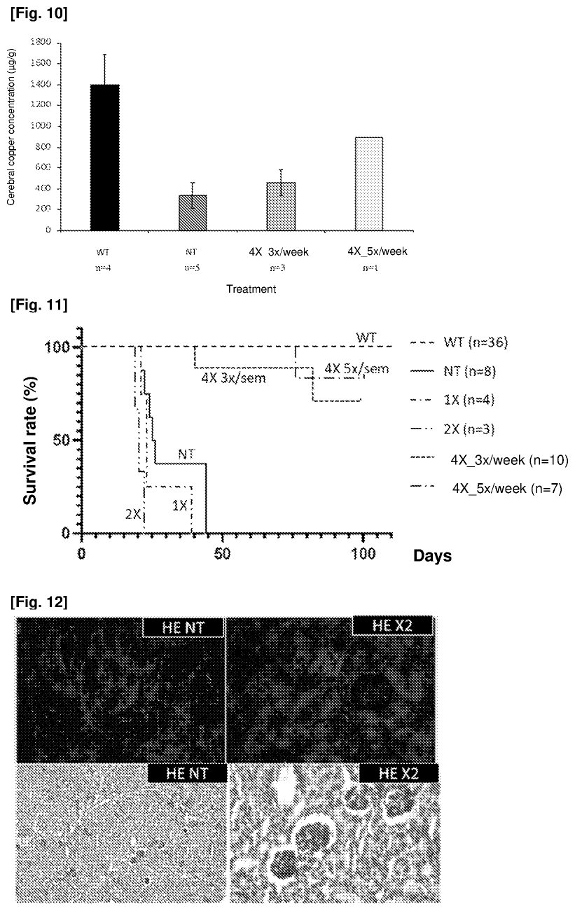

The results obtained for cerebral copper concentration are shown in FIG. 10. “Cerebral copper” is understood to mean the copper assayed in a whole brain homogenate. The copper is measured by atomic absorption spectrometry (PinAAcle 900T Atomic Absorption Spectrometer, Perkin Elmer). It can be seen from this figure that the cerebral copper concentration is correlated with the dosage of nanoclusters administered to the animals. The cerebral copper concentration increases with the increase of the dosage. The mice treated with the 4× dose, five days per week have a much larger cerebral copper concentration than those treated with 4× dose but three days per week.

### Life Expectancy of Hemizygous Mice

The results obtained for the survival of hemizygous mice are shown in FIG. 11. The animals that were untreated or treated with low doses (1× and 2×) have a lifetime which does not exceed 45 days whereas the animals with the 4× dose three days or five days per week have a greatly improved lifetime, because the more majority of these animals are still alive at over 100 days of life. Further, no adverse reaction was observed in the mice, including in the group treated with the 4× dose. No tissue anomaly was observed in the tissues studied after the animals were sacrificed.

### Analysis of the Motricity of Hemizygous Mice

A difference in the motor activity of the animals was seen between the wild mice and the hemizygous mice. With aging, the untreated hemizygous mice had a notable reduction of their overall activity (reduction of motor activity, atonia, impossibility of standing upright, etc.). In fact, the mice experienced some difficulty moving around and feeding. This is seen as a difficulty reaching the feeder with an impossibility of standing upright like the wild mice do.

When the hemizygous mice were treated with the copper nanocluster solution at the 1× dose, a progressive decrease of activity was seen, thus leading to a dominance of the wild type animals. In this case, the hemizygous mice were prostrate in a corner and did not move.

In contrast, in the group of hemizygous mice treated with the copper nanocluster solution at the 4× dose, a large improvement of the overall activity was seen. The mice treated with the 4× dose stood upright and jumped more easily. This shows an improvement of the physical shape of the mice treated with the copper nanocluster solution at the 4× dose.

### Analysis of the Fur Pigmentation by Optical Microscope

The fur is a good indicator of the metabolic state of the mice. The pigmentation of the fur, which is a highly observable aspect in hemizygous mice, is analyzed in the following. In hemizygous mice treated with a copper nanocluster solution at the 4× does, an alternation of dark and light zones on the fur is seen under optical microscope; this could correspond to the periodicity of the injections previously given. In fact, the administration of copper by means of the copper nanoclusters solution restores the production of melanin, because it depends on the activity of tyrosinase which is a cuproprotein. It is the improvement of this enzymatic function which is at the origin of the improvement of the coloring of the coat of the mice.

Staining of the Copper with Rhodanine: Preliminary Results

The anatomical pathology study of the copper content of the various tissues, by staining with rhodanine, served to show the absence of pathological accumulation of copper in the various tissues studied (liver, kidney, brain). With the goal of detecting the presence or absence of copper in the storage organs like the liver and kidney or in the organs where it is necessary for observing notable affects like the brain, the copper was marked using a rhodanine stain, with which to detect copper deposits within the target tissues. This study also served to identify a possible toxicity (in the liver and kidneys) by showing histological lesions of these organs related to the presence of copper in the hepatic and/or renal cells. These studies were done in hemizygous mice that were untreated and treated at the 2× copper nanocluster dose. The observed results are shown in FIGS. 12 (kidney) and 13 (liver).

The anatomical pathology analysis of the kidney (FIG. 12) shows the presence of small deposits of copper around the kidney of the animals treated with the 2× doses (top right figure) without there being any apparent tissue toxicity near the kidney tissue (bottom right figure).

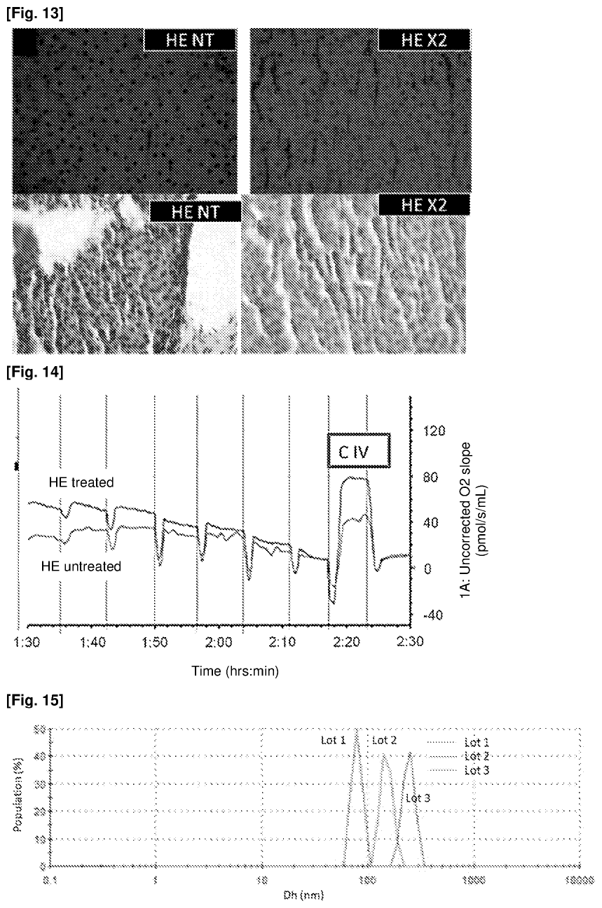

The anatomical pathology analysis of the liver (FIG. 13) show the presence of small deposits of copper around the liver of the animals treated with the 2× does (top right figure) without there being any apparent of tissue toxicity near the liver tissue (bottom right figure).

These data show that there is in fact capture of copper by the tissues, without leading to cellular toxicity in the kidney and the liver at the 2× treatment dose.

### Analysis of the Cerebral Respiratory Chain

Cellular respiration is one of the functions altered when there is a copper deficiency. It has been shown that a failure of insertion of copper in the respiratory chain leads to a defect of functionality of the complex I and complex IV and also a defect of the oxidative phosphorylation. In Menkes syndrome, the affected cerebral zones are principally the basal ganglia. Because of this, the mitochondrial respiration was measured in the stratum of mice included in the study (wild mice, untreated hemizygous mice, hemizygous mice treated at the dose 2×) by means for high resolution oxygen (Oroboros, O2k, Laboratoire NGERE). The results obtained are shown in FIG. 14. On this figure, a distinct improvement of the respiratory chain activity can be seen in the treated mice (upper curve) compared to the untreated mice (lower curve). This improvement is particularly visible around the complex IV (C IV) which comprises the copper-related proteins.

The analysis of the cerebral respiratory chain activity is an index of the restoration of activities related to the cerebral cuproproteins. In fact, the complex IV activity of the respiratory chain is dependent on the presence of functional cuproproteins. The restoration of this complex IV activity (FIG. 14) therefore shows that the copper nanoclusters allow penetration of copper into the brain, and that they do therefore pass the blood-brain barrier. The copper that is thus available can be used in the neurons for restoring a copper-dependent enzymatic activity.

### CONCLUSION

The in vivo tests done on the mice show that:
The treatment with the copper nanoclusters allows an improvement of the cerebral copper concentration in treated animals.
The increase of the cerebral copper concentration allows an improvement of the activity of complex IV of the respiratory chain which is dependent on the presence of copper.

These data show that the copper nanoclusters from the pension pass through the blood-brain barrier and that the coper is bioavailable for restoring the cerebral enzyme activity dependent on the presence of copper. Further the administration of copper nanoclusters participates in the survival of the treated animals.

### Example 4: Comparative Example

### Preparation of Copper Nanoclusters from Copper Chloride or Copper Sulfate

Inventors prepared copper nanoclusters by substituting copper acetate with copper chloride or copper sulfate. The protocol used is the solution protocol described in Example 1, the only difference lies in the use of copper chloride or copper sulfate in place of copper acetate.

Solutions of copper chloride 1× or copper sulfate 1× are respectfully prepared for getting respectively copper nanocluster solutions 1×.

The copper concentration of each of the resulting copper nanocluster solutions, possibly dialyzed, is 16 μg/mL.

The three solutions of copper nanoclusters 1× obtained respectively from copper acetate, copper chloride or copper sulfate are compared in terms of stability. The stability was evaluated by dynamic light diffusion as indicated in Example 2 above. The results obtained showed a much greater stability, by monitoring changes to the hydrodynamic diameter is a function of time, when the copper acetate is used in comparison to the copper chloride or sulfate. As indicated in the Example 2, the copper nanoclusters in solution from the invention have a stability ranging up to 140 days whereas the stability of the copper nanoclusters in solution resulting from copper chloride is only two days and that of copper nanoclusters in solution resulting from copper sulfate is only four days. In fact, by measuring the hydrodynamic diameter as a function of time, it increased a great deal (over 100 nm after two days or after four days when the copper nanoclusters are respectively prepared based on copper chloride or copper sulfate) thus showing the destabilization of copper nanoclusters.

### Comparison of Copper Nanoclusters from the Invention with the Copper Nanoclusters Described in the Document CN 105,965,028

Example 9 from the Chinese document describes the preparation of copper clusters from 0.01 mol/L of copper acetate, 0.1 mol/L of histidine and 0.1 mol/L of ascorbic acid. The protocol described in this example was reproduced identically by the inventors. The syntheses were done three times in total. The liquid mixtures of copper clusters obtained at the outcome of each of the three syntheses were respectively stored in three graduated test tubes with screwed stoppers and are called Lot 1, Lot 2 and Lot 3.

The hydrodynamic diameter (Dh) of the copper clusters from each of the lots were evaluated immediately after their synthesis, by dynamic light diffusion (angle 173°, laser 530 nm, temperature 25° C. on Malvern Nanosizer). The results obtained are shown in FIG. 15 where it can be seen that the hydrodynamic diameter of each of the three lots varies from 70 to 120 nm. Such hydrodynamic diameters therefore do not allow describing the clusters from the Chinese document as “nanoclusters.”

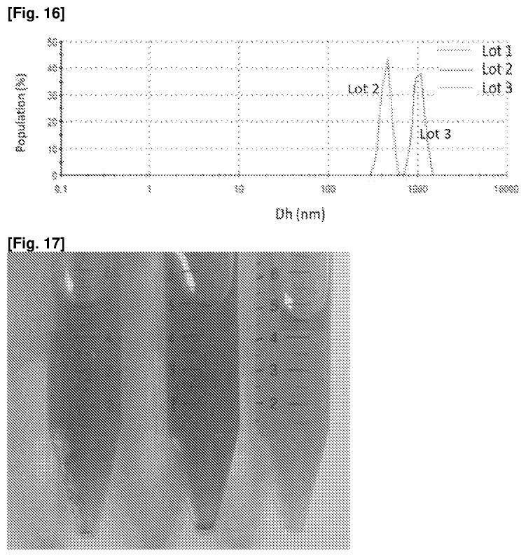

Lots 1 to 3 were then stored for 24 hours of 4° C. in the dark. The analysis of the hydrodynamic diameter (DH) was restarted at the end of 24 hours after their synthesis. The results obtained are shown in FIG. 16. Only two curves can be seen on this figure because Lot 1 is superimposed on Lot 2. It can be seen from FIG. 16 that the hydrodynamic diameter has greatly increased, which shows a bad stabilization of the clusters and a high tendency to aggregation. This finding of the bad stability and the aggregation is seen from a macroscopic point of view in FIG. 17 in the tubes in which the clusters are stored.

In conclusion, the resulting particles according to the protocol from the Chinese document are not nanoclusters (size greater than 2 nm) and are not stable.

The present disclosure is not limited to the examples described above, only as examples, but it encompasses all the variants which could be conceived by the person skilled in the art in the context of the protection sought.

1-15. (canceled)

16. Copper nanoclusters, wherein the copper nanoclusters:
are covered on the surface thereof by a mixed layer comprising histidine (His), acetate ions (Ac) and possibly ascorbate ions (Asc);
have a spherical shape;
have a hydrodynamical diameter ranging from 0.6 to 2.0 nm, preferably 1.3 nm;
have a metallic core with a diameter ranging from 0.5 to 1.5 nm, preferably 1.0 nm;
have a temporal stability ranging from 5 to 12 weeks, when the nanoclusters are in liquid form;
have a temporal stability of at least 12 months, and preferably of 12 to 18 months, when the nanoclusters are in dried form;
have spectrophotometric properties, with a shoulder in the UV-Visible spectrum at 325±10 nm and a fluorescence spectrum with excitation wavelengths ranging from 365 to 395 nm and emission wavelengths ranging from 445 to 475 nm;
said copper nanoclusters are designated by the formula “CuNC@HisAc” or by the formula “CuNC@HisAcAsc.”

17. The copper nanoclusters according to claim 16, wherein the copper nanoclusters are in liquid form or dry form.

18. The copper nanoclusters according to claim 16, wherein the copper nanoclusters have at least one of the following characteristics:
they are able to pass through the blood-brain barrier;
they have a good bioavailability;
they are biocompatible;
they are biodegradable;
they can be freeze-dried;
they are not toxic for the human body;
they do not accumulate in organs such as the liver, spleen, kidneys or lungs.

19. A method for preparation of copper nanoclusters according to claim 16, comprising the following steps:
reaction between copper (II) acetate and histidine in order to get a mixture of copper acetate and histidine, where the molar ratio of histidine to copper (II) acetate is greater than or equal to 50, and preferably goes from 50 to 200;
reaction between the mixture of copper acetate and histidine with ascorbic acid in order to get a mixture of copper acetate, histidine and ascorbic acid, where the molar ratio of ascorbic acid/copper (II) acetate is greater than or equal to 130, and preferably goes from 130 to 700;
recovery of copper nanoclusters covered on the surface thereof by a mixed layer comprising histidine, acetate ions and ascorbate ions.

20. The method according to claim 19, wherein the method is done under inert gas and:
the copper acetate is in solution form and the histidine is in powder form;
a solution of copper acetate and histidine is prepared by adding histidine to the copper acetate solution;
the copper acetate and histidine solution is adjusted to a pH value ranging from 11 to 13, and is preferably 12;
the ascorbic acid is in powder form;
a solution of copper acetate, histidine and ascorbic acid is prepared by addition of ascorbic acid to the copper acetate and histidine solution whose pH was adjusted to the aforementioned values;
the copper acetate, histidine and ascorbic acid solution is stirred for 2 hours to 6 hours, and preferably 4 hours, at a temperature ranging from 35° C. to 45° C., and preferably 40° C.;
a solution comprising copper nanoclusters is obtained at the outcome of the preceding stirring step;
the solution comprising the copper nanoclusters could be dialyzed in order to get a purified copper nanocluster solution;
the solution comprising the copper nanoclusters, possibly dialyzed, could be freeze-dried in order to get dry-form copper nanoclusters.

21. The method according to claim 20, further comprising at least one characteristic selected from:
the inert gas is nitrogen;
the copper acetate solution is prepared by addition of copper acetate to ultrapure filtered water;
the concentration of the copper acetate solution ranges from 0.5 to 5.0 mM;
the concentration of histidine is greater than the concentration of the copper acetate solution;
the pH of the copper acetate and histidine solution is adjusted using sodium hydroxide;
the ascorbic acid concentration is equal to the histidine concentration;
the solution comprising the copper nanoclusters, possibly dialyzed, has a copper concentration ranging from 16 to 160 μg/mL;
the solution comprising the copper nanoclusters, possibly dialyzed, is freeze-dried in order to get dry-form copper nanoclusters.

22. The method according to claim 19, wherein:
the copper acetate is in powder form and the histidine is in powder form;
a powdered mixture of copper acetate and histidine is obtained by mixing each of the powders of copper acetate and histidine;
the powdered copper acetate and histidine mixture is milled until obtaining a homogeneous color powdered mixture;
the homogeneous powdered copper acetate and histidine mixture is placed in a reaction vessel;
the ascorbic acid is in powder form;
ascorbic acid is added to the reaction vessel comprising the homogeneous powdered copper acetate and histidine mixture;
the resulting powdered copper acetate, histidine and ascorbic acid mixture is stirred, then water is added drop by drop to the reaction vessel, where said water is filtered ultrapure water;
the reaction vessel is placed under an inert gas and protected from light;
the copper acetate, histidine, ascorbic acid and water mixture is kept under stirring in the reaction vessel for 16 to 36 hours, preferably for 24 hours;
a liquid mixture comprising copper nanoclusters is obtained at the outcome of the preceding stirring step;
the liquid mixture comprising the copper nanoclusters could be dialyzed in order to get a purified liquid copper nanocluster mixture;
the liquid mixture comprising the copper nanoclusters, possibly dialyzed, could be freeze-dried in order to get dry-form copper nanoclusters.

23. The method according to claim 22, further comprising at least one characteristic selected from:
the concentration of histidine is greater than the concentration of copper acetate;
the ascorbic acid concentration is equal to the histidine concentration;
the water added to the reaction vessel is filtered ultrapure water;
the inert gas is nitrogen;
the liquid mixture comprising the copper nanoclusters, possibly dialyzed, has a copper concentration ranging from 1600 to 4000 μg/mL;
the liquid mixture comprising the copper nanoclusters, possibly dialyzed, is freeze-dried in order to get dry-form copper nanoclusters.

24. The method according to claim 20, wherein the dry-form copper nanoclusters are stored under nitrogen, preferably in a vial, and preferably at 4° C., where said powder may be stored for a length of at least 12 months, and preferably from 12 to 18 months, without alteration of the stability of the copper nanoclusters.

25. The copper nanoclusters as defined in claim 16 for use as medication.

26. A method of treating pathologies related to a copper deficit in a subject in a subject in need thereof, comprising administering to the subject a therapeutically effective amount of the copper nanoclusters as defined in claim 16.

27. The method according to claim 26, wherein the pathology related to a copper deficit is Menkes syndrome.

28. The method according to claim 27, wherein the copper nanoclusters are administered by subcutaneous administration, once per day.

29. The method according to claim 27, wherein the copper nanoclusters are administered by continuous administration via a subcutaneous pump.

30. The method according to claim 27, wherein the copper nanoclusters are administered to a pediatric subject.
